(19) United States
(12) Patent Application Publication
(10) Pub. No.: US 2013/0184544 A1
(43) Pub. Date: Jul. 18, 2013

(54) BODY-MOUNTED PHOTOACOUSTIC SENSOR UNIT FOR SUBJECT MONITORING

(71) Applicants: Mark Su, Boulder, CO (US); Kristi Cohrs, Englewood, CO (US); Friso Schlottau, Lyons, CO (US)

(72) Inventors: Mark Su, Boulder, CO (US); Kristi Cohrs, Englewood, CO (US); Friso Schlottau, Lyons, CO (US)

(73) Assignee: Nellcor Puritan Bennett LLC, Boulder, CO (US)

(21) Appl. No.: 13/349,650

(22) Filed: Jan. 13, 2012

(51) Int. Cl. A61B 5/145; A61B 6/00

(52) U.S. Cl. 600/323; 600/407; 600/322

(57) ABSTRACT

A body-mounted photoacoustic sensor unit may use photoacoustic sensing to determine one or more physiological parameters of a subject. The body-mounted photoacoustic sensor unit may fixably locate a light source and photoacoustic detector relative to a target area. The photoacoustic detector may detect an acoustic pressure response generated by the application and absorption of light from the light source.

The present disclosure relates to a body-mounted photoacoustic sensor unit.

### SUMMARY

A body-mounted photoacoustic sensor unit may have a light source, a photoacoustic detector, and a support assembly. The photoacoustic sensor unit may attach to a subject such that the light source and photoacoustic detector are fixably located relative to a target area such as a blood vessel. The light source may illuminate the target area, resulting in a pressure-wave or acoustic response that may be detected by the photoacoustic detector.

In some embodiments the body mounted photoacoustic sensor unit may comprise a processor coupled to the light source and the photoacoustic detector. The body mounted photoacoustic sensor unit may include a display for displaying information related to the operation of the photoacoustic sensor unit, e.g., status information or a value of a physiological parameter. The processor may be coupled to a transceiver, e.g., to communicate with a patient monitor over a wireless connection. A pressure sensor may detect a pressure of attachment of the body-mounted photoacoustic sensor unit to a subject. The body-mounted photoacoustic sensor unit may include a transport medium located between the target area and the photoacoustic detector. A power source may be coupled to the body mounted photoacoustic sensor unit, e.g., to provide power to the light source, photoacoustic detector, processor, display and/or transceiver.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative physiological monitoring system in accordance with some embodiments of the present disclosure;

FIG. 2 is a block diagram of an illustrative sensor unit of the physiological monitoring system of FIG. 1, which may be coupled to a subject in accordance with some embodiments of the present disclosure;

FIG. 3 shows a block diagram of an illustrative signal processing system in accordance with some embodiments of the present disclosure;

FIG. 4 is an illustrative photoacoustic arrangement in accordance with some embodiments of the present disclosure;

FIG. 5 is a plot of an illustrative photoacoustic signal, including two peaks corresponding to walls of blood vessels, in accordance with some embodiments of the present disclosure;

FIG. 6 is an illustrative perspective view of a wrist-mounted photoacoustic sensor unit in accordance with some embodiments of the present disclosure;

FIG. 7a is an illustrative perspective view of a photoacoustic sensor housing in accordance with some embodiments of the present disclosure;

FIG. 7b is an illustrative view of a photoacoustic sensor housing in accordance with some embodiments of the present disclosure;

FIG. 8 is an axial view of a wrist-mounted photoacoustic sensor unit showing a detector in proximity to the radial artery in accordance with some embodiments of the present disclosure;

FIG. 9 is an axial view of a wrist-mounted support assembly tab-and-slot attachment mechanism in accordance with some embodiments of the present disclosure; and

FIG. 10 is an exemplary display indicating parameters determined by a photoacoustic sensor in accordance with some embodiments of the present disclosure.

### DETAILED DESCRIPTION OF THE FIGURES

Photoacoustics, as used herein, describes the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for medical imaging, physiological parameter determination, or both. For example, the concentration of a constituent such as hemoglobin, both oxygenated and deoxygenated, blood pressure, pulse rate, and blood flow may be determined using photoacoustic analysis.

A photoacoustic system may include a photoacoustic sensor that is placed at a site on a subject, typically a wrist, palm, elbow, neck, forehead, temple, inner ear, thigh, or other location where blood vessels are within the sensitivity range of the instrument. The photoacoustic system may use a light source, and any suitable light guides (e.g., fiber optics), to pass light through the subject's tissue, or a combination of tissue thereof (e.g., organs), and an acoustic detector to sense the pressure response of the tissue. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's response to the incident light of the light source.

In some embodiments, the light from the light source may be focused, shaped, or otherwise spatially modulated to illuminate a particular target area. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated target area, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic (PA) signal. The PA signal may be used to calculate any of a number of physiological parameters, including an amount of a blood constituent (e.g., oxyhemoglobin), at a particular spatial location. In some embodiments, PA signals from multiple spatial locations may be used to construct an image (e.g., imaging blood vessels) or a scalar field (e.g., a hemoglobin concentration field).

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, for determining pulse rate or blood pressure, an infrared (IR) wavelength, for example 795 or 808 nm, may be used because it is sufficiently absorbed by blood. If additional physiological parameters are also determined using the photoacoustic system (e.g., oxygen saturation), Red and IR wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nano-second pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμ_{α}φ(z) (1)

under conditions where the irradiation time is small compared to the characteristic thermal diffusion time such as produced by a short-duration pulsed light source. Referring to Eq. 1, p(z) is the PA signal (indicative of the maximum induced pressure rise) at spatial location z indicative of acoustic pressure, Γ is the dimensionless Grüneisen parameter of the tissue, μₐ is the effective absorption coefficient of the tissue (or constituent thereof) to the incident light, and φ(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{P}} \qquad ( 2 )$$

where c_{α}² is the speed of sound in the tissue, β is the isobaric volume thermal expansion coefficient, and Cₚ is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{-μ}^{_{eff}}^{z} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured PA signal. In some embodiments, an ultrasound detector positioned relatively farther away from the region of interest, will encounter a relatively smaller acoustic pressure. For example, the acoustic pressure received at a circular area A_{d} positioned at a distance R from the illuminated region of interest may be given by Eq. 4:

p_{d}=p(z)f(rₛ,R,A_{d}) (4)

where rₛ is the radius of the illuminated region of interest (and typically rₛ<R), and p(z) is given by Eq. 1. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) \sim \frac{{p}_{0} ( {r}_{0} , ω )}{R} {e}^{- i ω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and τ is the travel time delay of the wave equal to R/c_{α}, where c_{α} is the speed of sound in the tissue. The FD-PA spectrum p₀(r₀,ω) of acoustic waves is shown illustratively by Eq. 6:

$${p}_{0} ( {r}_{0} , ω ) = \frac{Γ {μ}_{a} φ ( {r}_{0} )}{2 ( {μ}_{a} {c}_{a} - i ω )} . \qquad ( 6 )$$

where μ_{α}c_{α} represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t):

$$r ( t ) = cos ( t ( {ω}_{0} + \frac{b}{2} t ) ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with present disclosure. In some embodiments, signals having a characteristic frequency that changes as a nonlinear function of time may be used. Any suitable technique, or combination of techniques thereof, may be used to analyze a FD-PA signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a PA signal. As shown in Eq. 8:

$${B}_{s} ( t - τ ) = \frac{1}{2 π} {\int }_{- ∞}^{∞} H ( ω ) S ( ω ) {e}^{i ω t} d ω \qquad ( 8 )$$

Fourier transforms (and inverse transforms) are used to calculate the filter output Bₛ(t−T), in which H(ω) is the filter frequency response, S(ω) is the Fourier transform of the PA signal s(t), and T is the phase difference between the filter and signal. In some circumstances, the filter output of expression of Eq. 8 may be equivalent to an autocorrelation function. Shown in Eq. 9:

$$S ( ω ) = \frac{1}{2 π} {\int }_{- ∞}^{∞} s ( t ) {e}^{- i ω t} d t \qquad ( 9 )$$

is an expression for computing the Fourier transform S(ω) of the PA signal s(t). Shown in Eq. 10:

H(ω)=S*(ω)e^{-iωτ} (10)

is an expression for computing the filter frequency response H(ω) based on the Fourier transform of the PA signal s(t). It can be observed that the filter frequency response of Eq. 10 requires the frequency character of the PA signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) d {t}^{′} \qquad ( 11 )$$

the known modulation signal r(t) may be used for generating a cross-correlation with the PA signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The FD-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

$$cos ( A ) cos ( B ) = \frac{1}{2} [ cos ( A - B ) - cos ( A + B ) ] \qquad ( 12 )$$

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$L ( t ) = 〈 r ( t ) s ( t ) 〉 ≅ 〈 Kr ( t ) r ( t - \frac{R}{{c}_{a}} ) 〉 = \frac{1}{2} K cos ( \frac{R}{{c}_{a}} bt + θ ) \qquad ( 13 )$$

calculated by low-pass filtering (shown by angle brackets) the product of modulation signal r(t) and PA signal s(t). If the PA signal is assumed to be equivalent to the modulation signal, with a time lag R/c_{α} due to travel time of the acoustic wave and amplitude scaling K, then a convenient approximation of Eq. 13 may be made, giving the rightmost term of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/c_{α}. Assuming that the frequency scan rate b and the speed of sound c_{α} are known, the depth R may be estimated.

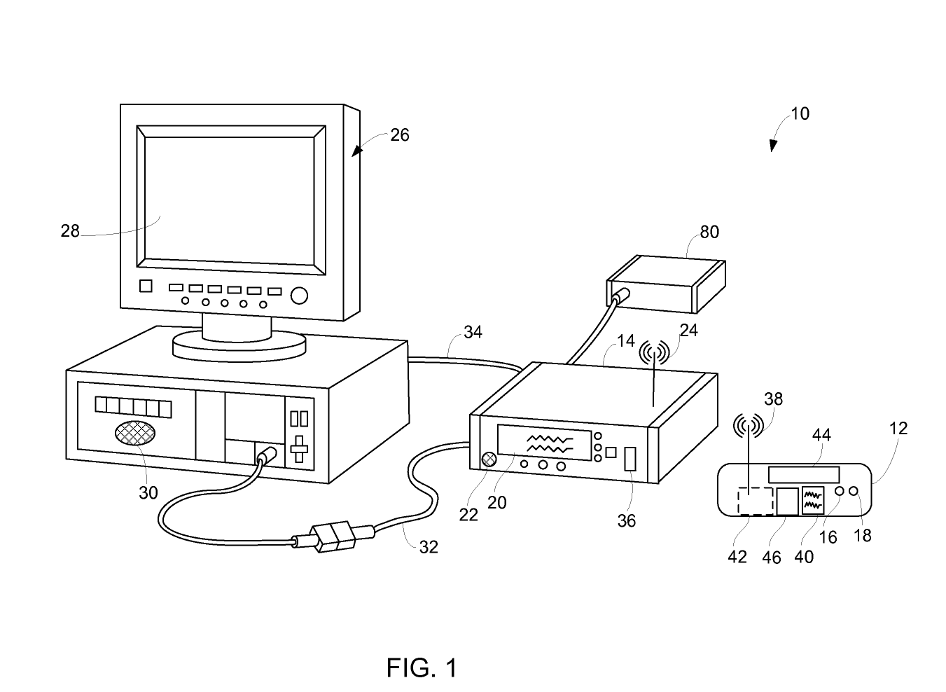

FIG. 1 is a perspective view of an embodiment of a physiological monitoring system 10 in accordance with some embodiments of the present disclosure. Physiological monitoring system 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue. A detector 18 may also be provided in sensor unit 12 for detecting the acoustic (e.g., ultrasound) response that emanates from the subject's tissue. Any suitable physical configuration of light source 16 and detector 18 may be used. In an embodiment, sensor unit 12 may include multiple light sources and/or detectors, which may be spaced apart. In an exemplary embodiment, sensor 12 may be a body-mounted sensor and may include wireless transceiver 38, display 40, processing circuitry 42, and power source 44.

Physiological monitoring system 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12 (e.g., a photoplethysmograph sensor). Multiple sensor units may be capable of being positioned at two or more different locations on a subject's body; for example, a first sensor unit may be positioned on a subject's wrist, while a second sensor unit may be positioned at a subject's fingertip, ankle, forehead, neck, or mouth.

In some embodiments, physiological monitoring system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, the sensor may be wirelessly connected to monitor 14 (e.g., via wireless transceivers 38 and 24) and include its own battery or similar power source 44. In some embodiments, sensor unit 12 may draw its power from monitor 14 and be communicate with monitor 14 via a physical connection such as a wired connection (not shown). Sensor unit 12, Monitor 14, or both, may be configured to calculate physiological parameters based at least in part on data relating to light emission and acoustic detection received at one or more sensor units such as sensor unit 12. For example, sensor unit 12, monitor 14, or both, may be configured to determine pulse rate, blood pressure, blood oxygen saturation (e.g., arterial, venous, or both), hemoglobin concentration (e.g., oxygenated, deoxygenated, or total), any other suitable physiological parameters, or any combination thereof. In some embodiments, some or all calculations may be performed on sensor unit 12 (i.e., using processing circuitry 42) or an intermediate device and the result of the calculations may be passed to monitor 14. Further, monitor 14 may include a display 20 configured to display the physiological parameters or other information about the system. Sensor unit 12 may also include a display 40 configured to display the physiological parameters or other information about the system and a user interface 46. In an exemplary embodiment, processing circuitry 42 may be configured to operate light source 16 and detector 18 to generate and process photoacoustic signals, communicate with display 40 to display values such as signal quality and power levels, receive signals from user input 46, and control wireless transceiver 38 to communicate data (e.g., photoacoustic output signals) with monitor 14.

In the embodiment shown, monitor 14 may also include a speaker 22 to provide an audible sound that may be used in various other embodiments, such as for example, sounding an audible alarm in the event that a subject's physiological parameters are not within a predefined normal range. In another embodiment, sensor unit 12 may communicate such information to the user, e.g., using display 40, an audible source such as a speaker, vibration, tactile, or any other means for communicating a status to a user, such as for example, in the event that a subject's physiological parameters are not within a predefined normal range.

In some embodiments, sensor unit 12 may be communicatively coupled to monitor 14 via a wireless system, utilizing antenna 38 of sensor unit 12 and antenna 24 of monitor 14. Antenna 38 may be external or internal to sensor unit 12, and capable of transmitting signals, receiving signals, or both transmitting and receiving signals, via amplitude modulated RF, frequency modulated RF, BLUETOOTH, IEEE 802.11, WiFi, WiMax, cable, satellite, infrared, any other suitable transmission scheme, or any combination thereof. Communication between the sensor unit 12 and monitor 14 may also be carried over a cable (not shown) to an input 36 of monitor 14, or to a multi-parameter physiological monitor 26 (described below). The cable may include electronic conductors (e.g., wires for transmitting electronic signals from detector 18, or a partially or fully processed signal from sensor unit 12), optical fibers (e.g., for transmitting emitted light from light source 16), any other suitable components, any suitable insulation or sheathing, or any combination thereof. Monitor 14 may include a sensor interface configured to receive physiological signals from sensor unit 12, provide signals and power to sensor unit 12, transfer data specific to the subject, general to the physiological parameter being measured, or both, or otherwise communicate with sensor unit 12. The sensor interface may include any suitable hardware, software, or both, which may be allow communication between monitor 14 and sensor unit 12.

In the illustrated embodiment, physiological monitoring system 10 includes multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a display 28 for information from sensor unit 12, monitor 14, or both, and from other medical monitoring devices or systems (not shown). For example, multi-parameter physiological monitor 26 may be configured to display an estimate of a subject's blood pressure, blood oxygen saturation, hemoglobin concentration, and/or pulse rate generated by sensor unit 12 or monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). The multi-parameter physiological monitor 26 may also be communicatively coupled to sensor unit 12 with or without the presence of monitor 14. Sensor unit 12 may be coupled to the multi-parameter physiological monitor 26 by a wireless connection using sensor unit antenna 38 and an antenna on the multi-parameter physiological monitor (not shown), or by a cable (not shown). In addition, sensor unit 12, monitor 14, or multi-parameter physiological monitor 26 may be coupled to a network to enable the sharing of information with servers or other workstations (not shown). In some embodiments this network may be a local area network, which may be further coupled through the Internet or other wide area network for remote monitoring. Sensor unit 12, monitor 14 and multi-parameter physiological monitor 26 may be powered by a battery (not shown) or by a conventional power source such as a wall outlet.

Calibration device 80, which may be powered by monitor 14, a battery, or by a conventional power source such as a wall outlet, may include any suitable calibration device for measuring a physiological parameter such as blood pressure. Although calibration device 80 is described herein as being attached to monitor 14, it will be understood that calibration device 80 could operate in any suitable manner, e.g., by attachment to sensor unit 12. Calibration device 80 may take the form of any invasive or non-invasive blood pressure monitoring or measuring system used to generate reference blood pressure measurements for use in calibrating the blood pressure monitoring techniques described herein. Such calibration devices may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference blood pressure measurements, or other physiological parameter, as needed, that are obtained from some other source (e.g., an external invasive or non-invasive blood pressure measurement system).

Calibration device 80 may also access reference blood pressure measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. As described in more detail below, the reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle. In the depicted embodiments, calibration device 80 is connected to monitor 14 via cable 82. In other embodiments, calibration device 80 may be a stand-alone device that may be in wireless communication with monitor 14. Reference blood pressure measurements may then be wirelessly transmitted to monitor 14 for use in calibration. In still other embodiments, calibration device 80 is completely integrated within monitor 14.

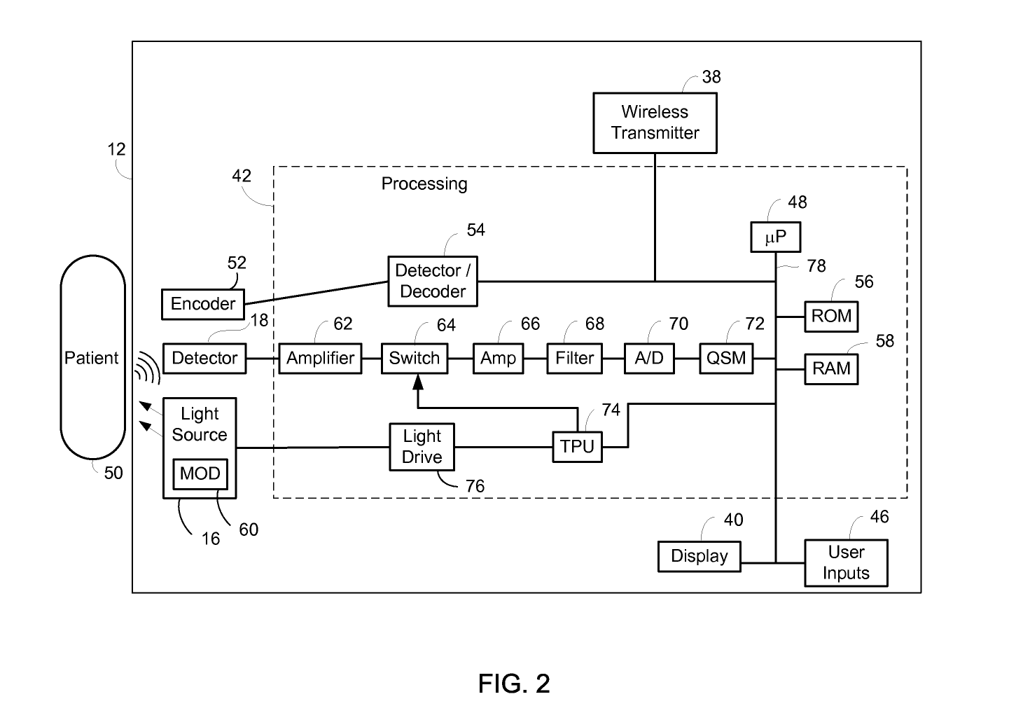

FIG. 2 is a block diagram of an illustrative sensor unit 12 of physiological monitoring system 10 of FIG. 1, which may be coupled to a subject 50 in accordance with some embodiments of the present disclosure. Although in certain embodiments sensor unit 12 may include the components depicted in FIG. 2, it will be understood that some components may be located external to sensor unit 12, e.g., certain aspects of processing circuitry 42.

Sensor unit 12 may include light source 16, detector 18, and encoder 52. In some embodiments, light source 16 may be configured to emit one or more wavelengths of light (e.g., visible, infrared) into a subject's tissue 50. Hence, light source 16 may provide Red light, IR light, any other suitable light, or any combination thereof, that may be used to calculate the subject's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor emits only a Red light while a second emits only an IR light. In a further example, the wavelengths of light used are selected based on the specific location of the sensor.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” shall refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure. In some embodiments, detector 18 may be a pressure-sensitive Faby-Pérot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using the piezoelectric effect, photodetector of a Faby-Pérot interferometer, or other suitable device). After converting the received acoustic pressure signal to an electrical signal, detector 18 may send the signal to processing circuitry 42, where physiological parameters may be calculated based on the photoacoustic activity within the subject's tissue 50. In another embodiment, processing circuitry 42 may process the received photoacoustic signal and transmit the processed signal to monitor 14 (e.g., using wireless transmitter 38), where physiological parameters may be calculated.

In some embodiments, encoder 52 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by processing circuitry 42 to select appropriate algorithms, lookup tables and/or calibration coefficients stored in processing circuitry 42 for calculating the subject's physiological parameters or processing the photoacoustic signal to be transmitted to monitor 14.

Encoder 52 may contain information specific to subject 50, such as, for example, the subject's age, weight, and diagnosis. This information about a subject's characteristics may allow processing circuitry 42 to determine, for example, subject-specific threshold ranges in which the subject's physiological parameter measurements should fall and to enable or disable additional physiological parameter algorithms. Encoder 52 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 52 may include a memory on which one or more of the following information may be stored for communication to processing circuitry 42: the type of the sensor unit 12; the wavelengths of light emitted by light source 16; the particular acoustic range that each sensor in the sensor array is monitoring; a signal threshold for each sensor in the sensor array; any other suitable information; or any combination thereof.

In some embodiments, signals from detector 18 and encoder 52 may be transmitted to processing circuitry 42. In the embodiment shown, processing circuitry 42 may include a general-purpose microprocessor 48 connected to an internal bus 78. Microprocessor 48 may be adapted to execute software, which may include an operating system and one or more applications, as part of performing the functions described herein. Also connected to bus 78 may be a read-only memory (ROM) 56, a random access memory (RAM) 58, user inputs 46, and display 40.

RAM 58 and ROM 56 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 74 may provide timing control signals to light drive circuitry 76, which may control the activation of light source 16. For example, TPU 74 may control pulse timing (e.g., pulse duration and inter-pulse interval) for TD-PA monitoring system. TPU 74 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 58 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 58, analog to digital converter 70, any other suitable component shown or not shown in FIG. 2) coupled by bus 78 or otherwise coupled (e.g., via an external bus), may be referred to as “processing equipment.”

In the embodiment shown, light source 16 may include modulator 60, in order to, for example, perform FD-PA analysis. Modulator 60 may be configured to provide intensity modulation, spatial modulation, any other suitable optical signal modulations, or any combination thereof. For example, light source 16 may be a CW light source, and modulator 60 may provide frequency modulation of the CW light source such as a linear frequency modulation. In some embodiments, modulator 60 may be included in light drive 76, or other suitable components of sensor unit 12, or any combination thereof.

In some embodiments, microprocessor 48 may determine the subject's physiological parameters, such as SpO₂, SvO₂, total hemoglobin concentration (t_{HB}), and/or pulse rate, using various algorithms and/or look-up tables based on the value of the received signals and/or data corresponding to the acoustic response received by detector 18. In other embodiments, microprocessor 48 may process a signal to be transmitted to monitor 14 (e.g., using wireless transmitter 38), where monitor 14 may determine the subject's physiological parameters. Signals corresponding to information about subject 50, and particularly about the acoustic signals emanating from a subject's tissue over time, may be transmitted from encoder 52 to decoder 54. These signals may include, for example, encoded information relating to subject characteristics. Decoder 54 may translate these signals to enable the microprocessor to determine the thresholds based at least in part on algorithms or look-up tables stored in ROM 56.

In some embodiments, user inputs 46 may be used to enter information, select one or more options, provide a response, input settings, any other suitable inputting function, or any combination thereof. User inputs 46 may be used to enter information about the subject, such as age, weight, height, diagnosis, medications, treatments, and so forth. In some embodiments, user inputs may be buttons, knobs, dials, a keyboard, mouse, touchpad, other suitable input device, or any combination thereof. User inputs may also be received from monitor 14, a multi-parameter physiological monitor such as 26 of FIG. 1, or a device coupled through a network. In some embodiments, display 40 may exhibit a list of values, which may generally apply to the subject, such as, for example, age ranges or medication families, which the user may select using user inputs 46.

The acoustic signal attenuated by the tissue of subject 50 can be degraded by noise, among other sources. Movement of the subject may also introduce noise and affect the signal. For example, the contact between the detector and the skin, or the light source and the skin, can be temporarily disrupted when movement causes either to move away from the skin. Another source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

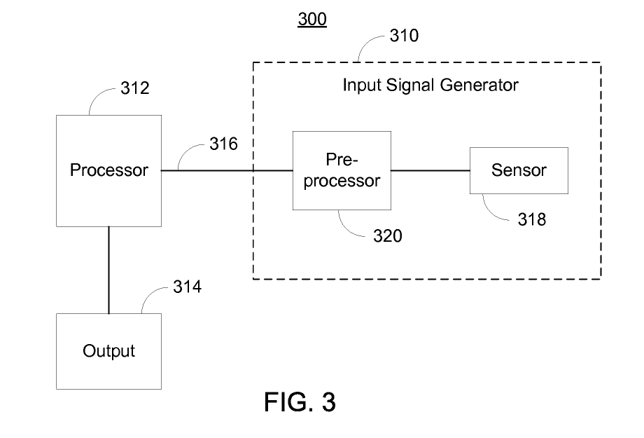

FIG. 3 shows a block diagram of an illustrative signal processing system 300 in accordance with some embodiments of the present disclosure. Signal processing system 300 may implement the signal processing techniques described herein. In some embodiments, aspects of signal processing system 300 may be implemented in a physiological monitoring system (e.g., physiological monitoring system 10 of FIGS. 1-2). It will be understood that the signal processing system 300 may be implemented in any suitable manner. In one embodiment, signal processing system 300 may be included in a single component of a physiological monitoring system (e.g., sensor unit 12 of physiological monitoring system 10 of FIGS. 1-2). In other embodiments, portions of processing system 300 may be contained in multiple components of a physiological monitoring system. For example, in one embodiment sensor unit 12 may perform the functionality of input signal generator 310 while monitor 14 performs the functionality of processor 312 and output 314.

In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In an embodiment, pre-processor 320 may be any suitable signal processing device and input signal 316 may include one or more PA signals and one or more other physiological signals, such as a photoplethysmograph signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as pulse rate, blood pressure, blood oxygen saturation (e.g., arterial, venous, or both), hemoglobin concentration (e.g., oxygenated, deoxygenated, or total), or any other suitable calculated values, in a memory device for later retrieval. Processor 312 may be coupled to a calibration device (not shown) that may generate or receive as input reference blood pressure measurements for use in calibrating blood pressure calculations.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

In some embodiments, portions of system 300 may be configured to be portable, e.g., as a body-mounted sensor unit 12. In another example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of physiological monitoring system 10 (FIGS. 1 and 2). As such, physiological monitoring system 10 (FIGS. 1 and 2) may be part of a fully portable and continuous physiological monitoring solution. In some embodiments, a wireless transceiver (not shown) may also be included in system 300 to enable wireless communication with other components of physiological monitoring system 10. For example, pre-processor 320 may output signal 316 over BLUETOOTH, IEEE 802.11, WiFi, WiMax, cable, satellite, infrared, any other suitable transmission scheme, or any combination thereof. In some embodiments, a wireless transmission scheme may be used between any communicating components of system 300.

It will also be understood that while some of the equations referenced herein are continuous functions, the processing equipment may be configured to use digital or discrete forms of the equations in processing the acquired PA signal.

In some embodiments, the illuminated region of interest may be a target area that includes a blood vessel such as an artery, vein, or capillary, for example. The blood within the vessel may absorb a portion of the incident optical fluence at the vessel. The resulting acoustic pressure signal may exhibit two sequential peaks (in the time domain) generated primarily from the boundary between the blood and the adjacent tissue (e.g., a blood vessel). The acoustic pressure signal, as detected at a suitable detector, may be greater when that boundary surface faces the detector. The first peak may be indicative of the front boundary between the blood and the vessel (relatively closer to the light source), and the second peak may be indicative of the back boundary between the blood and the vessel (relatively further from the light source).

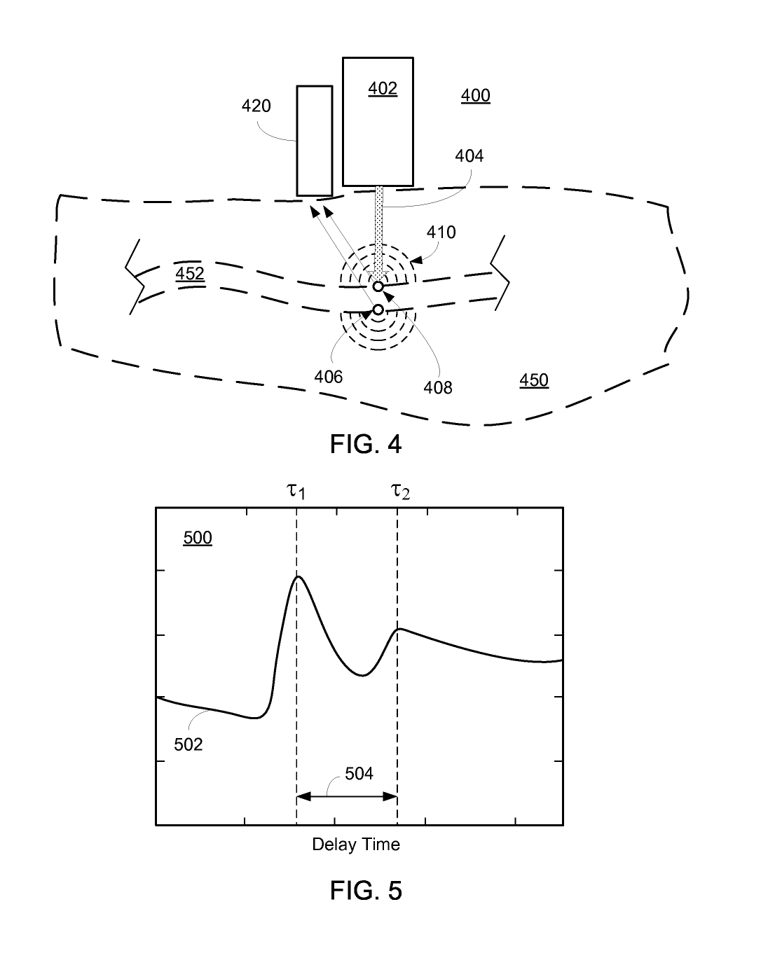

FIG. 4 is an illustrative photoacoustic arrangement 400 in accordance with some embodiments of the present disclosure. Light source 402, controlled by a suitable light drive (e.g., a light drive of system 300 or physiological monitoring system 10, although not shown in FIG. 4), may provide photonic signal 404 to subject 450. Photonic signal 404 may be attenuated along its pathlength in subject 450 prior to reaching blood vessel 452. A constituent of the blood in blood vessel 452 such as, for example, hemoglobin, may absorb at least some of photonic signal 404. Accordingly, the blood may exhibit an acoustic pressure response via the photoacoustic effect, which may act on the boundary of blood vessel 452. Acoustic pressure signals 410 may travel through subject 450, originating substantially from the front boundary 408 and back boundary 406 of blood vessel 452. Acoustic detector 420 (i.e., a photoacoustic detector) may detect acoustic pressure signals 410 traveling through tissue of subject 450, and output (not shown) a photoacoustic signal that may be processed. Because the path length between point 408 and acoustic detector 420 is shorter than the pathlength between point 406 and acoustic detector 420, it may be expected that acoustic pressure signals from point 408 may reach acoustic detector 420 before acoustic pressure signals from point 406. Additionally, in some arrangements, because the path length between point 408 and acoustic detector 420 is shorter than the path length between point 406 and acoustic detector 420, it may be expected that an acoustic pressure signal from point 408 may exhibit a relatively larger peak than an acoustic pressure signal from point 406. Accordingly, acoustic detector 420 may detect two sequential peaks in acoustic pressure signal 410 generated by photonic signal 404 directed at blood vessel 452.

FIG. 5 is a plot 500 of an illustrative photoacoustic signal 502, including two peaks corresponding to walls of blood vessels, in accordance with some embodiments of the present disclosure. The abscissa of plot 500 is presented in units proportional to time (e.g., delay time relative to a light pulse), while the ordinate of plot 500 is presented in arbitrary units of signal intensity. At least a portion of photoacoustic signal 502 corresponds to the acoustic pressure response of blood within a blood vessel. Photoacoustic signal 502 exhibits a first peak and a second peak, located at respective times τ₁ and t₂. The first peak corresponds to the front boundary of the blood vessel, relatively nearer to the acoustic detector. The second peak corresponds to the back boundary of the blood vessel, relatively further from the acoustic detector. It will be understood that the blood vessel diameter may also be determined by measuring other locations of the blood vessel, such as the change in the right to left sides of the blood vessels when suitable acoustic sensors are used. Time difference 504 between τ₁ and τ₂ indicates the relative difference in delay time between acoustic pressure signals from the front and back boundaries. The signal intensity may correspond to the absorption of the particular constituent of the target area. In some embodiments, analysis of the first and second peaks may allow the determination of one or more physiological parameters. When measuring the blood pressure in an artery, the distance between the two peaks will vary over the cardiac cycle and the different distances may correspond to different pressures.

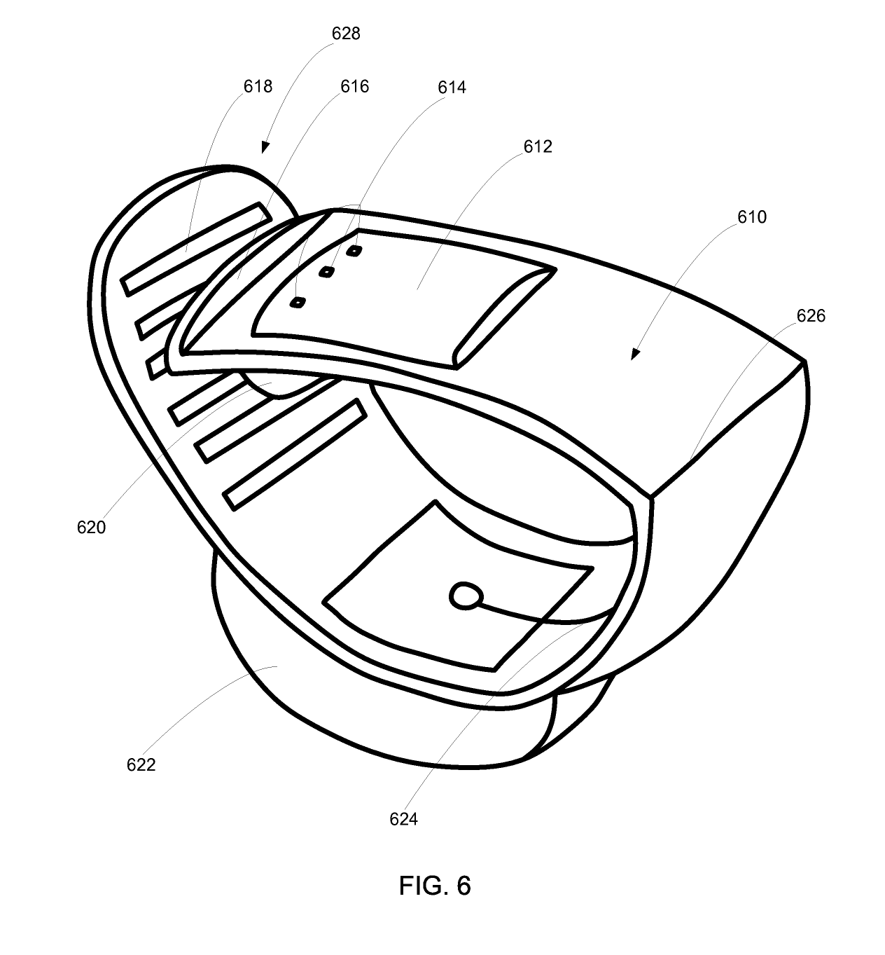

FIG. 6 is an illustrative perspective view of a wrist-mounted photoacoustic sensor unit in accordance with some embodiments of the present disclosure. It will be understood that the wrist is one of several possible locations that a body mounted sensor could be located to determine a physiological parameter of a subject. A support assembly of the body mounted sensor could also encircle an ankle, arm, neck, any other suitable location, or any suitable combination of locations. In some embodiments, the support assembly may be mounted to the subject without fully encircling a body part, but rather attach with an adhesive or other means such that the detector is located in proximity to a target area of the subject.

In the embodiment shown in FIG. 6, the support assembly 610 is a strap, band, or bracelet that will support the parts comprising a photoacoustic system such as sensor unit 12. The support assembly 610 comprises a support body and an attachment mechanism 628 which may be used to secure the support assembly 610 to the subject. The support assembly 610 may be constructed of any suitable material or combination of materials, such as rubber, plastic, polymer, metal, fabric, or any other suitable material.

Attachment mechanism 628 may be any suitable mechanism to secure support assembly 610 to a subject, such as tab-and-slot, snap, button, adhesive, hook-and-loop, buckle, or elastic connector. In some embodiments, the circumference of support assembly 610 may be adjusted by attachment mechanism 628 such that the pressure applied by sensing unit 620 to the subject may be adjusted.

A tab-and-slot mechanism is illustrated in the embodiment shown in FIG. 6, wherein tab 616 passes through one of slots 618 in order to secure the support assembly 610 to the subject. Multiple slots are provided to allow adjustment of the band and thus adjustment of the pressure applied by the sensor unit to the subject. In some embodiments, an angled section 626 of support assembly 610 is included which may function in part to maintain the security of the attachment mechanism 628.

In some embodiments of support assembly 610, a display 612 may be coupled to the sensor unit. This display may include a plurality of display elements 614, which may be LEDs of one or more colors, multi-color LEDs, incandescent, electroluminescent, incandescent bulbs, liquid crystal devices, electroluminescent devices, cathodoluminescent devices, electrophoretic devices, electrochromic devices, photoluminescent devices, or mechanical devices. Display 612 and display elements 614 may be controlled in any suitable manner, e.g., by processing circuitry 42 of FIG. 2. Display 612 may also include some or all of the functionality of user interface 46 of FIG. 2, such as one or more buttons, a touch screen, a jog control, or any other suitable interface. In some embodiments, a user interface may be located separately from display 612 (not shown), e.g., elsewhere at the wrist mounted sensor unit or at monitor 14 of FIG. 1.

A sensor unit 620 including a light source and detector may be coupled to the support assembly 610. Sensor unit 620 may provide a surface for locating the sensor relative to a target area, for example, on the anterior of the wrist.

A processing unit 622 may be coupled to the support assembly 610. In some embodiments, this unit will in part carry out some or all of the processing steps of processing circuitry 42 of FIG. 2, using data from sensor unit 620 and displaying results on display 612. Processing unit 622 may include a power source (not shown), it may be powered remotely, or a separate power source may be coupled to the support assembly. Cable 624 may be used to couple processing unit 622 to the sensor unit 620, display 612, or both.

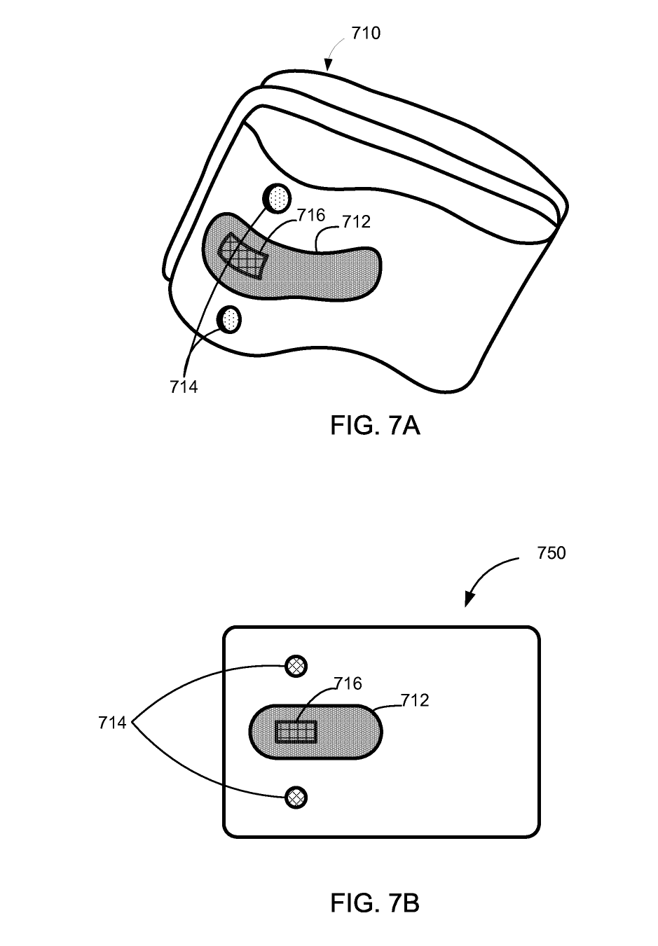

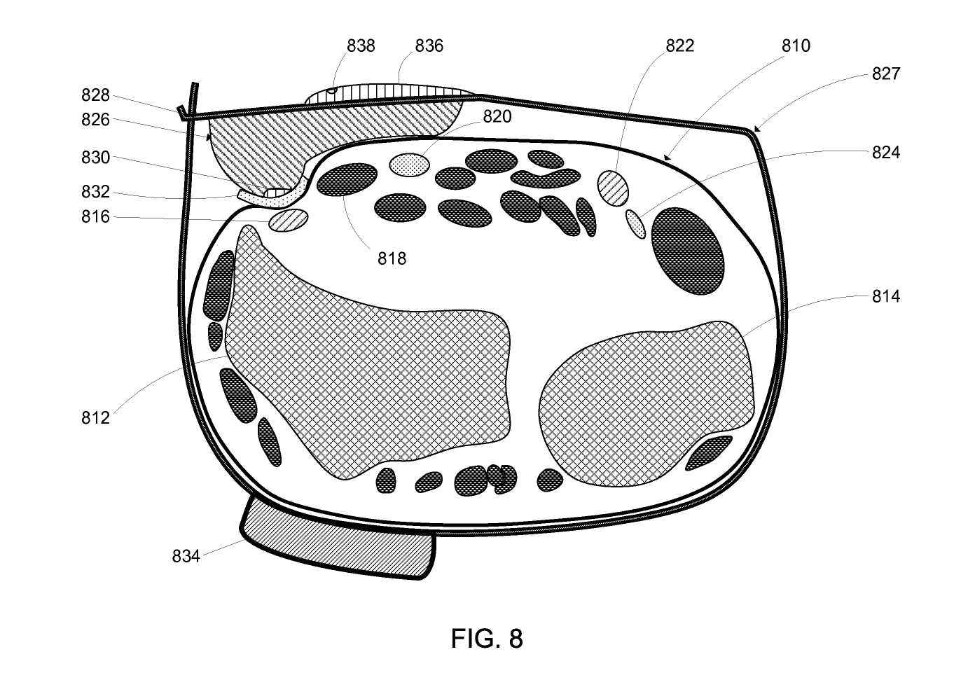

FIGS. 7a and 7b are two illustrative views of a photoacoustic sensor housing 710, e.g., of sensor unit 620 in accordance with some embodiments of the present disclosure. Sensor housing 710 may have any suitable shape to locate one or more light sources 714 and photoacoustic detector 716 relative to a target area. In the exemplary embodiment depicted by the perspective view of the sensor housing in FIG. 7a, a contoured shape may be configured to locate the sensor relative to a target area of the subject. As depicted, the contoured shape in section 718 is convex and the contoured shape in section 720 is concave. This shape may facilitate the placement and alignment of the sensor on the wrist as shown in FIG. 8.

Sensor housing 710 may include one or more light sources 714 to illuminate a target area. In an exemplary embodiment, two light sources 714 are shown, but it will be understood that any number of light sources may be arranged in any suitable manner used to generate a photoacoustic signal. In some embodiments, the light source may be separated from the detector or detector array by a distance between 0.2 mm and 40 mm. In an exemplary embodiment, a plurality of light sources may be employed successively at some switching rate in order to avoid heating of the skin while at the same time maintaining the photoacoustic signal used to determine at least one physiological parameter of the subject. In another exemplary embodiment, an array of light sources may surround the detector, allowing for multiple wavelengths to be implemented and avoiding local heating of the subject.

In FIG. 7b, sensor housing 710 is shown in an illustrative top view. An acoustic transport medium 712 may be interposed between a target area and photoacoustic detector 716. Photoacoustic detector 716 may be configured as a linear array of ultrasound detectors, a 2-dimensional or 3-dimensional array of detectors, or in any other suitable manner. In some embodiments, detector 716, acoustic transport medium 712, or both may be tuned to any appropriate range of acoustic frequencies.

Acoustic transport medium 712 may be any suitable material that allows for the efficient acoustic coupling and transfer of a photoacoustic response signal from a subject to detector 716, such as a hydrogel, colloid, hydrocolloid, adhesive, polymer, gel, or liquid. Acoustic transport medium 712 may displace the air gap between the tissue and the sensor such that acoustic transport medium 712 may improve coupling of the photoacoustic response in the subject and the photoacoustic detector. Acoustic transport medium 712 may be impedance matched with the photoacoustic response within the subject, thereby reducing signal degradation and increasing signal power transfer. In some embodiments, a flexible acoustic transport medium 712 may help mitigate changes in the nature of the photoacoustic detector contact when the subject moves. The acoustic transport medium may have an adhesive applied to one or both sides to further strengthen the interaction with either or both the detector and the subject.

In some embodiments, sensor housing 710 may also include a pressure sensor (not shown) to indicate the pressure applied where the detector abuts the subject. The pressure sensor may detect the pressure using mechanical techniques, electronic techniques, or a combination thereof. A pressure sensor may provide an indication of the level of photoacoustic coupling, i.e., of proper attachment to the patient. A low pressure level may result in degraded performance, e.g., due to a failure to align with a target area or poor acoustic coupling. A high pressure level may be indicative of a pressure that may impede, reduce, or otherwise alter blood flow and other physiological processes within or near the target area. Data from a pressure sensor may be displayed on a display, e.g., display 612 of FIG. 6, or transmitted to an external monitor, e.g., monitor 14 of FIG. 1.

FIG. 8 is an axial view of a wrist-mounted photoacoustic sensor unit showing the detector in proximity to the radial artery in accordance with some embodiments of the present disclosure. It will be understood that a sensor unit may be located relative to a target area in any suitable manner, and that the embodiment depicted in FIG. 8 is exemplary only. Region 810 depicts an exemplary cross-section of a subject's wrist proximate to a target area for measurement by a photoacoustic sensor. The radius bone of the forearm 812 and the smaller ulna 814 are within the forearm region 810. In one embodiment, a target area may be radial artery 816, which may be located proximal to the radius 812. In another embodiment, a target area may be the ulnar artery 822, located proximal to ulna 814. Flexor carpi radialis 818 is located proximal to the radial artery in the wrist, and may extend from the elbow to the bones of the hand. In an exemplary embodiment, radius 812 and flexor carpi radialis 818 may be used to seat or otherwise locate sensor 826 relative to radial artery 816. In the embodiment described in FIG. 8, the sensor may be aligned with a target area of radial artery 816.

Support assembly 827 (e.g., support assembly 610 of FIG. 6) is depicted in an axial view. Attachment mechanism 828 (e.g., attachment mechanism 628 of FIG. 6) is illustrated as a tab and slot mechanism in this embodiment. Sensor body 826 may be coupled to support assembly 827 such that detector 830 is located proximate to radial artery 816. As shown, the contoured shape of sensor body 826 may assist in seating and locating detector 830.

Acoustic transport medium 832 (e.g., acoustic transport medium 712 of FIG. 7) is depicted in the axial view of FIG. 8. The flexible material of acoustic transport medium 832 may conform to the surfaces formed by both the sensor body 826 and the arm of the subject 810 under the pressure applied by the sensor body, filling any air gaps and improving acoustic transport of the acoustic signal generated in the radial artery 816 to the detector 830.

Detector 830 may receive a photoacoustic response from radial artery 816 and provide a signal to processing unit 834. Processing unit 834 may perform suitable processing steps to calculate a physiological parameter or process a signal to be provided to another device (e.g., monitor 14 of FIG. 1) for additional processing. Processing unit 834 may communicate with display unit 836 (having indicator lights 838) and/or a wireless transceiver (not shown) to communicate information to a user.

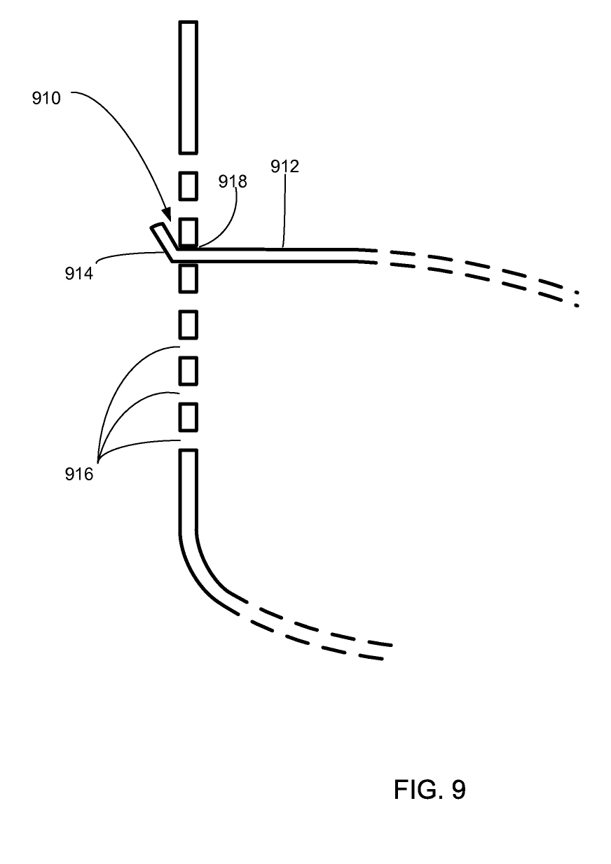

FIG. 9 is an axial view of a wrist-mounted support assembly tab-and-slot attachment mechanism in accordance with some embodiments of the present disclosure. As shown in FIG. 9, a tab-and-slot attachment mechanism may be used to secure the support assembly to the subject with a desired amount of pressure. A first end of the support assembly 912 may have a tab 914 embodied by a shaped or molded hook, barb, or section substantially rigid and substantially angled away from the general plane of 912, such that the tab prevents 912 disengagement with slot 918.

In some embodiments, tension may be generated by support assembly based on the circumference of the support assembly in relation to the circumference of the attachment area, e.g., a wrist. The tension may secure the assembly to the subject based on pressure applied by the sensor unit to the subject. Multiple slots 916 may be provided to enable adjustment of the circumference of the support assembly, thereby permitting the tension of the support assembly and applied pressure to be adjusted.

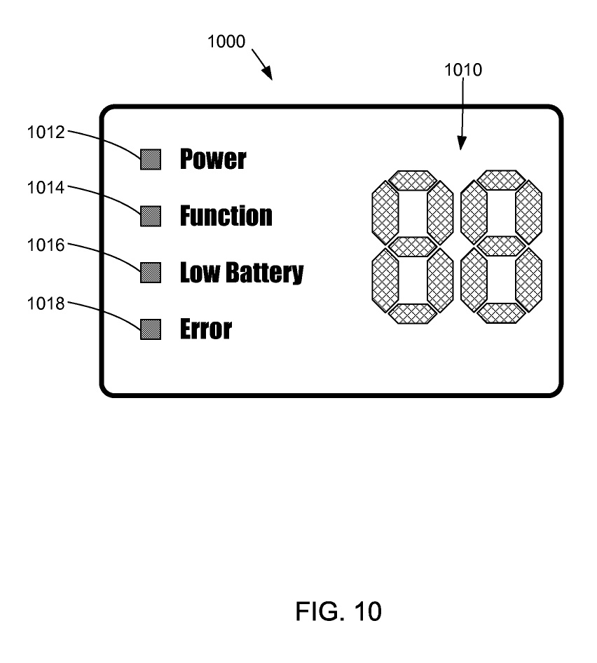

FIG. 10 is an exemplary display 1000 indicating parameters determined by a photoacoustic sensor in accordance with some embodiments of the present disclosure. Display 1000 may be any suitable display as described herein. In an exemplary embodiment, 4 single color LED lights 1012, 1014, 1016, and 1018 may provide relevant indications regarding the operation of the body-mounted sensor unit. For example, LED 1012 may indicate whether the power is on, LED 1014 may indicate whether the device is functioning properly, LED 1016 may indicate whether the battery power is low, and LED 1018 may indicate whether an error has occurred. The number, position, and nature of the indicator lights may be as many and of whatever type and arrangement is suitable. In some embodiments, one or more LEDs may indicate an alarm when, for example, a subject's physiological parameter exceed a threshold.

Display 1000 may also include any suitable display such as a 7 segment LED display 1010, e.g., to indicate a physiological parameter of the subject as determined by the sensor. This display may be configured to display pulse rate, blood pressure, blood oxygen saturation (e.g., arterial, venous, or both), hemoglobin concentration (e.g., oxygenated, deoxygenated, or total), any other suitable physiological parameters, or any combination thereof. In some embodiments, display 1010 may display additional information such as physiological trend information, battery level information, alarm information, information aiding the placement and positioning of the sensor unit on a subject, any other suitable information, and any combination thereof.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A body-mounted photoacoustic sensor unit comprising:
a light source, wherein the light source is configured to transmit light into a subject, and wherein the transmitted light is capable of generating a photoacoustic response in the subject;
a photoacoustic detector configured to detect the photoacoustic response; and
a support assembly configured to secure the photoacoustic sensor unit to the subject and coupled to the light source and photoacoustic detector, wherein the light source and photoacoustic detector are fixably located relative to a target area of the subject.

2. The photoacoustic sensor unit of claim 1, wherein the light source comprises one or more laser diodes and wherein the wavelength of the one or more laser diodes is between 600 nm and 1000 nm.

3. The photoacoustic sensor unit of claim 1, wherein the photoacoustic detector comprises a linear piezoelectric ultrasound receiver array of at least one dimension.

4. The photoacoustic sensor unit of claim 1, further comprising an acoustic transport medium coupled to the support assembly, wherein the acoustic transport medium is located at least partially between the target area and the photoacoustic detector.

5. The photoacoustic sensor unit of claim 4, wherein the acoustic transport medium comprises a hydrogel, colloid, or hydrocolloid.

6. The photoacoustic sensor unit of claim 4, wherein the acoustic transport medium is impedance matched to the photoacoustic response of the target area.

7. The photoacoustic sensor unit of claim 1, wherein the target area comprises a blood vessel proximal to the skin of the subject.

8. The photoacoustic sensor unit of claim 7, wherein the target area comprises a blood vessel from at least one of a wrist, ankle, neck, temple, inner ear, and thigh.

9. The photoacoustic sensor unit of claim 7, wherein the support assembly comprises a surface with a convex abutment that aligns the photoacoustic detector on the subject with the blood vessel.

10. The photoacoustic sensor unit of claim 9, wherein the convex abutment is configured to substantially align the photoacoustic detector between the flexor carpi radialis muscle and radius bone of the forearm, to detect the photoacoustic response of the radial artery.

11. The photoacoustic sensor unit of claim 1, wherein the support assembly comprises a support body and an attachment mechanism.

12. The photoacoustic sensor unit of claim 11, wherein the support body comprises a flexible strap.

13. The photoacoustic sensor unit of claim 11, wherein the attachment mechanism comprises a tab-and-slot, hook-and-loop, or an adhesive.

14. The photoacoustic sensor unit of claim 1, further comprising a display coupled to the support assembly.

15. The photoacoustic sensor unit of claim 14, wherein the display is configured to indicate a location of the target area, a functional state of the system, patient specific information, a power status of the system, an error status of the system, or a physiological parameter.

16. The photoacoustic sensor unit of claim 14, wherein the display comprises a plurality of light emitting diodes.

17. The photoacoustic sensor unit of claim 1, further comprising a pressure sensor coupled to the support assembly, wherein the pressure sensor is configured to determine the pressure applied by the photoacoustic sensor unit to the subject.

18. The photoacoustic sensor unit of claim 1, further comprising a processor configured to determine a physiological parameter based on an output signal of the photoacoustic detector.

19. The photoacoustic sensor unit of claim 18, wherein the physiological parameter comprises blood pressure, blood solute concentration, blood flow, or blood oxygenation.

20. The photoacoustic sensor unit of claim 18, further comprising a memory coupled to the processor and the support assembly.

21. The photoacoustic sensor unit of claim 18, further comprising a transceiver coupled to the processor and the support assembly.

22. The photoacoustic sensor unit of claim 18, further comprising a power source coupled to the processor and the support assembly.
